(19) United States
(12) Patent Application Publication
(10) Pub. No.: US 2005/0037354 A1
(43) Pub. Date: Feb. 17, 2005

(54) Method for epigenetic knowledge generation

(76) Inventors: Kurt Berlin, Stahnsdore (DE); Aron Braun, Berlin (DE); Peter Adorjan, Berlin (DE)

Correspondence Address: KRIEGSMAN & KRIEGSMAN, 665 FRANKLIN STREET, FRAMINGHAM, MA, 01702, US

(21) Appl. No.: 10/494,123

(22) PCT Filed: Oct. 25, 2002
(86) PCT No.: PCT/EP02/11960

Related U.S. Application Data
(60) Provisional application No. 60/334,708, filed on Oct. 31, 2001.

(51) Int. Cl. C12Q 1/68; G06F 15/18; G06F 19/00; G01N 33/48; G01N 33/50

(52) U.S. Cl. 435/6; 702/19; 706/14

(57) ABSTRACT

A method for epigenetic knowledge generation which designs and synthesizes the chemical and/or biological components that determine the epigenetic parameters to be selected and measured is described. The value of these epigenetic parameters is determined, the steps of this procedure repeated and finally the results are stored. The present invention relates to a method of epigenetic knowledge generation comprising the steps of: a. selecting epigenetic parameters of interest; b. designing the chemical and/or biological components of the epigenetic measurement system, wherein the chemical and/or biological components determine the epigenetic parameters to be measured; c. synthesizing the variable chemical and/or biological components; d. measuring the value of the epigenetic parameters using the chemical and/or biological components; e. storing the results obtained by measurement; f. defining a subset of epigenetic parameters of interest based on the measurements; g. repeating steps a-d.

In the context of the present invention, “epigenetic parameters” are, in particular, cytosine methylations and further chemical modifications of DNA bases of genes associated with DNA adducts and sequences further required for their regulation. Further epigenetic parameters include, for example, the acetylation of histones which, however, cannot be directly analyzed using the described method but which, in turn, correlate with the DNA methylation.

Molecular portraits, such as mRNA expression or DNA methylation patterns, have been shown to be strongly correlated with phenotypical parameters. These molecular patterns can be revealed routinely on a genomic scale. However, class prediction based on these patterns is an under-determined problem, due to the extreme high dimensionality of the data compared to the usually small number of available samples. This makes a reduction of the data dimensionality necessary. By comparing several feature selection methods, the right dimension reduction strategy is of crucial importance for the classification performance.

In recent years there has been a large interest in the analysis of mRNA expression by using microarrays (Lockhart, D. J., Winzeler, E. A., “Genomics, gene expression and DNA arrays.” Nature 405:827-836 (2000). This technology makes it possible to look at thousands of genes, see how they are expressed as proteins and gain insight into cellular processes. An important and scientifically interesting application of this technology is the classification of tissue types (Golub, T. R., et al. “Molecular classification of cancer: Class discovery and class prediction by gene expression monitoring.” Science 286:531-537 (1999); Ben-Dor, A., et al. “Tissue classification with gene expression profiles.” RECOMB01, in press (2001); Weston J., et al. “Feature Selection for SVMs.” To appear in Advances in neural information processing systems 13. MIT Press, Cambridge, Mass. (2001)).

However, there are some practical problems with the large scale analysis of mRNA based microarrays. They are primarily impeded by the instability of MRNA (Emmert-Buck, T., et al. “Molecular profiling of clinical tissue specimens: feasibility and applications.” Am J Pathol. 156:1109-15 (2000). Also expression changes of only a minimum of a factor 2 can be routinely and reliably detected. Furthermore, sample preparation is complicated by the fact that expression changes occur within minutes following certain triggers. The inability to resolve the individual contributions of such influences on an expression profile, and difficulties with quantifying the gradual nature of the occurring changes complicates data analysis.

An alternative approach is to look directly at DNA methylation. Methylation is a modification of cytosine in the combination CpG that can occur either with or without a methyl group attached. The methylated CpG can be seen as a 5th base and is one of the major factors responsible for expression regulation (Robertson, K. D., Wolffe, A. P., “DNA methylation in health and disease.” Nature Reviews Genetics 1:11-19 (2000). Aberrant DNA methylation within CpG islands is common in human malignancies leading to abrogation or overexpression of a broad spectrum of genes. Abnormal methylation has also been shown to occur in in CpG rich regulatory elements in intronic and coding parts of genes for certain tumors.

5-Methylcytosine is the most frequent covalent base modification in the DNA of eukaryotic cells. Therefore, the identification of 5-methylcytosine as a component of genetic information is of considerable interest. However, 5-methylcytosine positions cannot be identified by sequencing since 5-methylcytosine has the same base pairing behavior as cytosine. Moreover, the epigenetic information carried by 5-methylcytosine is completely lost during PCR amplification.

A relatively new and currently the most frequently used method for analyzing DNA for 5-methylcytosine is based upon the specific reaction of bisulfite with cytosine which, upon subsequent alkaline hydrolysis, is converted to uracil which corresponds to thymidine in its base pairing behavior. However, 5-methylcytosine remains unmodified under these conditions. Consequently, the original DNA is converted in such a manner that methylcytosine, which originally could not be distinguished from cytosine by its hybridization behavior, can now be detected as the only remaining cytosine using “normal” molecular biological techniques, for example, by amplification and hybridization or sequencing. All of these techniques are based on base pairing which can now be fully exploited. In terms of sensitivity, the prior art is defined by a method which encloses the DNA to be analyzed in an agarose matrix, thus preventing the diffusion and renaturation of the DNA (bisulfite only reacts with single-stranded DNA), and which replaces all precipitation and purification steps with fast dialysis (Olek A, Oswald J, Walter J. A modified and improved method for bisulphite based cytosine methylation analysis. Nucleic Acids Res. 1996 December 15;24(24):5064-6). Using this method, it is possible to analyze individual cells, which illustrates the potential of the method. However, currently only individual regions of a length of up to approximately 3000 base pairs are analyzed, a global analysis of cells for thousands of possible methylation events is not possible. However, this method cannot reliably analyze very small fragments from small sample quantities either. These are lost through the matrix in spite of the diffusion protection.

An overview of the further known methods of detecting 5-methylcytosine may be gathered from the following review article: Rein, T., DePamphilis, M. L., Zorbas, H., Nucleic Acids Res. 1998, 26, 2255.

To date, barring few exceptions (e.g., Zeschnigk M, Lich C, Buiting K, Doerfler W, Horsthemke B. A single-tube PCR test for the diagnosis of Angelman and Prader-Willi syndrome based on allelic methylation differences at the SNRPN locus. Eur J Hum Genet. 1997 March-April; 5(2):94-8) the bisulfite technique is only used in research. Always, however, short, specific fragments of a known gene are amplified subsequent to a bisulfite treatment and either completely sequenced (Olek A, Walter J. The preimplantation ontogeny of the H19 methylation imprint. Nat Genet. 1997 November; 17(3):275-6) or individual cytosine positions are detected by a primer extension reaction (Gonzalgo M L, Jones P A. Rapid quantitation of methylation differences at specific sites using methylation-sensitive single nucleotide primer extension (Ms-SNuPE). Nucleic Acids Res. 1997 June 15;25(12):2529-31, WO Patent 9500669) or by enzymatic digestion (Xiong Z, Laird P W. COBRA: a sensitive and quantitative DNA methylation assay. Nucleic Acids Res. 1997 June 15;25(12):2532-4). In addition, detection by hybridization has also been described (Olek et al., WO 99 28498).

Further publications dealing with the use of the bisulfite technique for methylation detection in individual genes are: Grigg G, Clark S. Sequencing 5-methylcytosine residues in genomic DNA. Bioessays. 1994 June; 16(6):431-6, 431; Zeschnigk M, Schmitz B, Dittrich B, Buiting K, Horsthemke B, Doerfler W. Imprinted segments in the human genome: different DNA methylation patterns in the Prader-Willi/Angelman syndrome region as determined by the genomic sequencing method. Hum Mol Genet. 1997 March; 6(3):387-95; Feil R, Charlton J, Bird A P, Walter J, Reik W. Methylation analysis on individual chromosomes: improved protocol for bisulphite genomic sequencing. Nucleic Acids Res. 1994 February 25;22(4):695-6; Martin V, Ribieras S, Song-Wang X, Rio M C, Dante R. Genomic sequencing indicates a correlation between DNA hypomethylation in the 5′ region of the pS2 gene and its expression in human breast cancer cell lines. Gene. 1995 May 19;157(1-2):261-4; WO 97 46705, WO 95 15373 and WO 45560.

An overview of the Prior Art in oligomer array manufacturing can be gathered from a special edition of Nature Genetics (Nature Genetics Supplement, Volume 21, January 1999), published in January 1999, and from the literature cited therein.

Fluorescently labeled probes are often used for the scanning of immobilized DNA arrays. The simple attachment of Cy3 and Cy5 dyes to the 5′-OH of the specific probe are particularly suitable for fluorescence labels. The detection of the fluorescence of the hybridized probes may be carried out, for example via a confocal microscope. Cy3 and Cy5 dyes, besides many others, are commercially available.

Genomic DNA is obtained from DNA of cell, tissue or other test samples using standard methods. This standard methodology is found in references such as Fritsch and Maniatis eds., Molecular Cloning: A Laboratory Manual, 1989.

By the term “individual” is meant, for the purposes of the specification and claims to refer to any mammal, especially humans.

### DESCRIPTION

No matter which biological platform technology or data-source will dominate the future health-care industry, there will by far be no product in such demand as tools for storage, administration, organization, secure transfer and the interpretation of complex epigenetic data. In particular, when the focus of the sector turns from blue-print data to information on the epigenetics of individuals, an explosion of available data will result, unprecedented in industry.

The optimal strategy involves intelligently setting up broad screens and then quickly narrowing those to the relevant parameters. It requires creating a short feedback loop from the interpretation of experimental results to the definition of the next series of experiments. Such an approach will be flexible enough to meet the demands of pharmaceutical research for not only more data, but for more relevant information.

This invention, an epigenetic knowledge generation method builds up a strong technological infrastructure that allows the tapping of classical diagnostic procedures for the integration with epigenetic data. This method consists of the following six steps:

In the first step, the epigenetic parameters of interest are selected. In a preferred embodiment, CpG sites from selected genes are analyzed.

Preferably, DNA extracted from all samples is enzymatically digested and bisulphite treated, converting all unmethylated cytosines to uracil whereas methylated cytosines are conserved.

In the second step, chemical and/or biological components of the epigenetic measurement system are designed. These chemical and/or biological components determine the epigenetic parameters to be measured. Preferably, PCR primers are designed complementary to DNA segments containing no CpG dinucleotides. This allows unbiased amplification of both methylated and unmethylated alleles in one reaction. In a preferred embodiment, regions of interests are then amplified by PCR using fluorescently labelled primers converting originally unmethylated CpG dinucleotides to TG and conserving originally methylated CpG sites.

In the third step, the variable chemical and/or biological components are synthesized. Preferably, a substrate to which DNA synthesis linkers have been applied with a temporarily protected surface is used as a solid support for the probes that are to be assembled. Preferably, to activate the surface of the substrate to couple the first level of bases, a high precision light image is projected onto the surface, illuminating only those areas of the surface of the substrate which are to bind a first base. Even more preferably, the projection of the image is performed by the use of electronically addressable micromirrors (DE 19922942.2 and DE 19932487.5).

Preferably, in the areas of the array exposed to light free hydroxy groups are formed which are capable of binding the appropriate base. Preferably, after this protection step a fluid containing the appropriate base is provided to the active surface of the substrate and the selected base binds to the exposed and thereby active sites. Preferably, the process is then repeated to bind another base to a different set of areas, until all the elements of the array on the substrate surface have an appropriate base of the first level of bases bound thereto. Preferably, the bases bound on the substrate are temporarily protected with a chemical capable of being removed under illumination and a new image is then projected onto the substrate to activate the protected surface in those areas to which the first base of the next level of bases is to be added. Preferably, a solution containing the selected base is applied to the array so that the base binds to the exposed areas. Preferably, this process is then repeated for all of the other areas of the second level of bases. Preferably, the process as described may then be repeated for each desired level of bases until the entire selected array of probe sequences has been completed. In a preferred embodiment, the array of sequences is finally entirely deprotected.

In the fourth step, the value of the epigenetic parameters is measured using the chemical and/or biological components. Preferably, all PCR products performed on an individual sample are mixed and hybridized to glass slides carrying for each CpG position a pair of immobilized oligonucleotides. Preferably, each of the detection oligonucleotides was designed to hybridize to the bisulphite converted sequence around one CpG site which was originally unmethylated (TG) or methylated (CG). Preferably, hybridization conditions were selected to allow the detection of the single nucleotide differences between the TG and CG variants. Preferably, ratios for the two signals were calculated based on comparison of intensity of the fluorescent signals. Preferably, the sensitivity of the method for detection of methylation changes was determined using artificially up- and downmethylated DNA fragments mixed at different ratios. Preferably, for each of those mixtures, a series of experiments was conducted to define the range of CG/TG ratios that corresponds to varying degrees of methylation at each of the CpG sites tested.

In the fifth step, the results obtained by measurement are stored. Preferably, this is done in a computing device, or transferred to a computing device from another computing device, storage device or hard copy, when the information has been previously determined. Preferably, the interpreted information integrated from different sources are amendable for storage in one unified framework.

In the sixth step, a subset of epigenetic parameters of interest is defined based on the measurements.

In the seventh step, the steps one to five are repeated. Preferably, this involves the management of enormous amounts of data.

Preferably, the steps one to seven of the epigenetic knowledge generation method are distributed among several locations. The data, chemical and/or biological components in question are preferably shipped in a systematic way between the units implementing any of the steps involved.

For the epigenetic knowledge generation method the design of the chemical and/or biological components of the epigenetic measurement system, the synthesis of the variable chemical and/or biological components and the measurement of the value of the epigenetic parameters is preferably integrated into a single device. This device preferably consists of the input interface for the design specification, the unit for synthesizing the desired chemical and/or biological components that can be varied in the process and that are determined by the specification of the epigenetic parameters of interest, the unit for measurement and the interface for transmitting the measurement results towards the component that interprets the experimental results.

In a preferred embodiment, the epigenetic parameters of interest for the epigenetic knowledge generation method comprise the methylation status of a single or a plurality of CpG dinucleotids in the genome.

In another preferred embodiment, the epigenetic parameters of interest for the epigenetic knowledge generation method comprise the methylation status of CpG dinucleotids within selected fragments of selected genes.

Preferably, the epigenetic parameters of interest for the epigenetic knowledge generation method comprise the methylation status of CpG dinucleotids within promoter regions of selected genes. Even more preferably, the epigenetic parameters of interest for the epigenetic knowledge generation method comprise the methylation status of CpG islands in selected genes.

In a preferred embodiment, the chemical and biological components of the epigenetic measurement system are determined such that the measured set of epigenetic parameters is identical to the selected epigenetic parameters of interest for the epigenetic knowledge generation method.

In another preferred embodiment, the chemical and biological components of the epigenetic measurement system are determined such that the measured set of epigenetic parameters differs from the selected epigenetic parameters of interest for the epigenetic knowledge generation method up to a predefined extent.

In still another embodiment, the difference between the epigenetic parameters of interest for the epigenetic knowledge generation method and the epigenetic parameters to be measured is estimated.

Preferably, the steps of selecting epigenetic parameters of interest for the epigenetic knowledge generation method, designing the chemical and/or biological components of the epigenetic measurement system and synthesizing the variable chemical and/or biological components are repeated until a predefined data quality is obtained.

Preferably, the selection of epigenetic parameters of interest for an epigenetic knowledge generation method involves queries in a knowledge representation system that contains known correlations between genetic and/or epigenetic and phenotypic parameters.

In a preferred embodiment, the epigenetic parameters of interest for the epigenetic knowledge generation method are tightened or broadened interactively.

In another preferred embodiment, the epigenetic parameters of interest for the epigenetic knowledge generation method contain epigenetic parameters with known or unknown function.

In another aspect of the invention, the invention provides a computer program product for an epigenetic knowledge generation method that includes a) means for selecting epigenetic parameters of interest using a computer readable program code; b) means for designing the chemical and/or biological components of the epigenetic measurement system, wherein the chemical and/or biological components determine the epigenetic parameters to be measured, using a computer readable program code; c) means for synthesizing the variable chemical and/or biological components using a computer readable program code; d) means for measuring the value of the epigenetic parameters using the chemical and/or biological components using a computer readable program code; e) means for storing the results obtained by measurement using a computer readable program code; f) defining a subset of epigenetic parameters of interest based on the measurements using a computer readable program code and g) repeating steps a-d.

Preferably, the steps a-g of the computer program product of the epigenetic knowledge generation method are distributed among several locations and the data, the chemical and/or biological components are shipped in a systematic way between the units implementing any of these steps.

For the computer program product of the epigenetic knowledge generation method the design of the chemical and/or biological components of the epigenetic measurement system, the synthesis of the variable chemical and/or biological components and the measurement of the value of the epigenetic parameters is preferably integrated into a single device. This device consists of the input interface for the design specification, the unit for synthesizing the desired chemical and/or biological components that can be varied in the process and that are determined by the specification of the epigenetic parameters of interest, the unit for measurement and the interface for transmitting the measurement results towards the component that interprets the experimental results.

In a preferred embodiment, the epigenetic parameters of interest for the computer program product of the epigenetic knowledge generation method comprise the methylation status of a single or a plurality of CpG dinucleotids in the genome.

In another preferred embodiment, the epigenetic parameters of interest for the computer program product of the epigenetic knowledge generation method comprise the methylation status of CpG dinucleotids within selected fragments of selected genes.

Preferably, the epigenetic parameters of interest for the computer program product of the epigenetic knowledge generation method comprise the methylation status of CpG dinucleotids within promoter regions of selected genes. Even more preferably, the epigenetic parameters of interest for the computer program product of the epigenetic knowledge generation method comprise the methylation status of CpG islands in selected genes.

In a preferred embodiment, the chemical and biological components of the epigenetic measurement system are determined such that the measured set of epigenetic parameters is identical to the selected epigenetic parameters of interest for the computer program product of the epigenetic knowledge generation method.

In another preferred embodiment, the chemical and biological components of the epigenetic measurement system are determined such that the measured set of epigenetic parameters differs from the selected epigenetic parameters of interest for the computer program product of the epigenetic knowledge generation method up to a predefined extent.

In still another embodiment, the difference between the epigenetic parameters of interest for the computer program product of the epigenetic knowledge generation method and the epigenetic parameters to be measured is estimated.

Preferably, the selection of epigenetic parameters of interest for the computer program product of an epigenetic knowledge generation method involves queries in a knowledge representation system that contains known correlations between genetic and/or epigenetic and phenotypic parameters.

In a preferred embodiment, the epigenetic parameters of interest for the computer program product of the epigenetic knowledge generation method are tightened or broadened interactively.

In another preferred embodiment, the epigenetic parameters of interest for the computer program product of the epigenetic knowledge generation method contain epigenetic parameters with known or unknown function.

In another aspect of the invention, the invention provides a system for epigenetic knowledge generation that includes a) means for selecting epigenetic parameters of interest using a computer readable program code; b) means for designing the chemical and/or biological components of the epigenetic measurement system, wherein the chemical and/or biological components determine the epigenetic parameters to be measured, using a computer readable program code; c) means for synthesizing the variable chemical and/or biological components using a computer readable program code; d) means for measuring the value of the epigenetic parameters using the chemical and/or biological components using a computer readable program code; e) means for storing the results obtained by measurement using a computer readable program code; f) means for defining a subset of epigenetic parameters of interest based on the measurements and g) repeating steps a-d.

Preferably, the steps a-g of the system for epigenetic knowledge generation are distributed among several locations and the data, the chemical and/or biological components are shipped in a systematic way between the units implementing any of these steps.

For the system of epigenetic knowledge generation the design of the chemical and/or biological components of the epigenetic measurement system, the synthesis of the variable chemical and/or biological components and the measurement of the value of the epigenetic parameters is preferably integrated into a single device. This device consists of the input interface for the design specification, the unit for synthesizing the desired chemical and/or biological components that can be varied in the process and that are determined by the specification of the epigenetic parameters of interest, the unit for measurement and the interface for transmitting the measurement results towards the component that interprets the experimental results.

In a preferred embodiment, the epigenetic parameters of interest for the system of epigenetic knowledge generation comprise the methylation status of a single or a plurality of CpG dinucleotids in the genome.

In another preferred embodiment, the epigenetic parameters of interest for the system of epigenetic knowledge generation comprise the methylation status of CpG dinucleotids within selected fragments of selected genes.

Preferably, the epigenetic parameters of interest for the system of epigenetic knowledge generation comprise the methylation status of CpG dinucleotids within promoter regions of selected genes. Even more preferably, the epigenetic parameters of interest for the system of epigenetic knowledge generation comprise the methylation status of CpG islands in selected genes.

In a preferred embodiment, the chemical and biological components of the epigenetic measurement system are determined such that the measured set of epigenetic parameters is identical to the selected epigenetic parameters of interest for the system of epigenetic knowledge generation.

In another preferred embodiment, the chemical and biological components of the epigenetic measurement system are determined such that the measured set of epigenetic parameters differs from the selected epigenetic parameters of interest for the system of epigenetic knowledge generation up to a predefined extent.

In still another embodiment, the difference between the epigenetic parameters of interest for the system of epigenetic knowledge generation and the epigenetic parameters to be measured is estimated.

Preferably, the selection of epigenetic parameters of interest for the system of epigenetic knowledge generation involves queries in a knowledge representation system that contains known correlations between genetic and/or epigenetic and phenotypic parameters.

In a preferred embodiment, the epigenetic parameters of interest for the system of epigenetic knowledge generation are tightened or broadened interactively.

In another preferred embodiment, the epigenetic parameters of interest for the system of epigenetic knowledge generation contain epigenetic parameters with known or unknown function.

The information generated can be translated into knowledge-based guidelines for physicians.

### EXAMPLE 1

Epigenetic parameters are obtained by treating genomic DNA with bisulphite. Prior to this modification the DNA is enzymatically digested with MSSI.

For the PCR amplification of the bisulphite treated sense strand of the 11 genes used the primers are designed. CpG sites from the following genes are analyzed: ELK1, CSNK2B, MYCL1, CD63, CDC25A, TUBB2, CD1 A, CDK4, MYCN, AR, c-MOS. The template DNA (10 ng), 12.5 pmol of each primer (Cy5-labelled), 0.5-2 U Taq polymerase and 1 mM dNTPs are incubated in the reaction buffer supplied with the enzyme in a total volume of 20 μl.

After activation of the enzyme (15 min, 96° C.) the incubation times and temperatures are 95° C. for 1 min followed by 34 cycles (95° C. for 1 min, annealing temperature for 45 sec, 72° C. for 75 sec) and 72° C. for 10 min.

Afterwards the oligonucleotides with a C6-amino modification at the 5′-end are spotted with 4-fold redundancy on activated glass slides. For each analyzed CpG position two oligonucleotides, reflecting the methylated and non methylated status of the CpG dinucleotides, are spotted and immobilized on the glass array.

The oligonucleotide microarrays representing 81 CpG sites are hybridized with a combination of up to 11 Cy5-labeled PCR fragments. The fluorescent images of the hybridized slides are obtained using a GenePix 4000 microarray scanner and directly entered into a database.

On a set of selected CpG sites statistical methods are applied. The CpG sites are ranked for a given separation task. The significance of each CpG for this separation task is estimated by a two sample t-test or alternatively by calculating the Fisher score (Bishop, C. M., Oxford University Press, New York (1995). All CpG sites with significance smaller p=0.05 are selected.

Based on the software applied, the circle from experimental design to data generation, evaluation and interpretation to the design of the next experiment is closed and models of cell function continuously refined to aid in the design of new DNA chip experiments for methylation detection.

### EXAMPLE 2

Sample preparation, bisulfite treatment and PCR amplification are performed as described in Example 1. The PCR products are hybridized to in situ synthesized oligomer arrays, that are produced as described in: Weiler et al. Nucleic Acids Research, 1997, 25, 2792, or as described in: Singh-Gasson et al. Nature Biotechnology, 1999, 17, 974. The Hybridisation conditions are adapted to give optimal performance for the required mismatch detection. The scanning of the arrays is performed as described in Example 1 and the gathered data is also processed the same way. The advantage of using in situ synthesized arrays is their cost advantage over arrays of presynthesized oligos when only small numbers of equal arrays are required and a significant reduction of turn around time.

### EXAMPLE 3

Cell development and cell differentiation associated genomic methylation patterns are continually being investigated. However, to use the detection of CpG methylation patterns as a genetic marker, the specific location and methylation status of CpG positions within relevant genes is required to be assessed. These analyses need to be performed in all the different cell kinds and cell states of interest, covering a broad range from highly differentiated, biologically functioning cells to completely undifferentiated stem or progenitor cells, before the gene's suitability as a marker can be evaluated.

For the search of sets of marker candidates other possible methods are the following. Differential methylation hybridization, Restriction landmark genomic scanning, Methylation sensitive AP-PCR and Methylated CpG island amplification all allow the identification of individual CpG positions which have a different methylation status in each of the classes under investigation. CpG positions thereby identified are herein referred to as Methylation Sequence Tag (MeST).

Identification of CpG islands may also be carried out using one or more of several restriction enzyme based methods. Such methods, allow the analysis of global genomic methylation patterns for which sequence information is unavailable. Alternatively candidate CpG positions may be identified using literature searches of journals, or by use of online databases in order to identify genes of interest associated with CpG island. Furthermore, where sequence information is available analysis of CpG positions may be carried out using bisulphite based technologies.

For this experiment tissue samples were taken from patients treated with Tamoxifen as an adjuvant therapy immediately following surgery. Samples were representative of the target population and as unbiased as possible.

The genomic DNA was isolated from the cell samples. It is required that the genomic DNA is from as pure a source as possible. The isolated genomic DNA from the samples was treated using a bisulfite solution (hydrogen sulfite, disulfite).

The treated nucleic acids were then amplified using multiplex PCRs of a large selection of genes, amplifying several fragments per reaction with fluorescently labeled primers.

All PCR products from each individual sample were then hybridized to glass slides carrying a pair of immobilized oligonucleotides for each CpG position under analysis. Each of these detection oligonucleotides was designed to hybridize to the bisulphite converted sequence around one CpG site which was either originally unmethylated (TG) or methylated (CG). Hybridization conditions were selected to allow the detection of the single nucleotide differences between the TG and CG variants.

Fluorescent signals from each hybridized oligonucleotide were detected. Ratios for the two signals (from the CG oligonucleotide and the TG oligonucleotide used to analyze each CpG position) were calculated based on comparison of intensity of the fluorescent signals.

The data obtained is then sorted into a ranked matrix according to CpG methylation differences between the tissues, using an algorithm.

For selected distinctions, a learning algorithm (support vector machine, SVM) was trained. The SVM (as discussed by F. Model, P. Adorjan, A. Olek, C. Piepenbrock, Feature selection for DNA methylation based cancer classification. Bioinformatics. 2001 June;17 Suppl 1:S157-64) constructs an optimal discriminant between two classes of given training samples. In this case each sample is described by the methylation patterns (CG/TG ratios) at the investigated CpG sites.

The SVM was trained on a subset of samples, which were presented with the diagnosis attached. Independent test samples, which were not shown to the SVM before were then presented to evaluate, if the diagnosis can be predicted correctly based on the predictor created in the training round. This procedure was repeated several times using different partitions of the samples, a method called crossvalidation.

All rounds were performed without using any knowledge obtained in the previous runs. The number of correct classifications was averaged over all runs.

The best oligonucleotides out of this process that produce informative results and a further selection of candidate oligonucleotides (which are suspected of being informative) are tested a multiple number of times. Therefore the whole procedure is repeated, i.e. PCR amplification, chip hybridization, data generation, evaluation and interpretation, until the marker genes are optimized.

In order to deduce the methylation status of the CpG positions, the CpG methylation information for each patient sample treated with Tamoxifen was collated and then used for further analyses.

1. A method of epigenetic knowledge generation comprising the steps of:
a. selecting epigenetic parameters of interest;
b. designing the chemical and/or biological components of the epigenetic measurement system, wherein the chemical and/or biological components determine the epigenetic parameters to be measured;
c. synthesizing the variable chemical and/or biological components;
d. measuring the value of the epigenetic parameters using the chemical and/or biological components;
e. storing the results obtained by measurement;
f. defining a subset of epigenetic parameters of interest based on the measurements;
g. repeating steps a-d.

2. A method according to claim 1, where steps a-f are distributed among several locations and wherein the data, the chemical and/or biological components are shipped in a systematic way between the units implementing any of these steps.

3. A method according to claim 1, where steps b, c and d are integrated into a single device comprising:
a. the input interface for the design specification;
b. the unit for synthesizing the desired chemical and/or biological components that can be varied in the process and that are determined by the specification of the epigenetic parameters of interest;
d. the unit for measurement;
e. the interface for transmitting the measurement results towards the component that interprets the experimental results.

4. A method according to any of the claims 1, 2 or 3, wherein the epigenetic parameters of interest comprise the methylation status of a single or a plurality of CpG dinucleotids in the genome.

5. A method according to any of the claims 1, 2 or 3, wherein the epigenetic parameters of interest comprise the methylation status of CpG dinucleotids within selected fragments of selected genes.

6. A method according to any of the claims 1, 2 or 3, wherein the epigenetic parameters of interest comprise the methylation status of CpG dinucleotids within promoter regions of selected genes.

7. A method according to any of the claims 1, 2 or 3, wherein the epigenetic parameters of interest comprise the methylation status of CpG islands in selected genes.

8. A method according to claim 1, wherein the chemical and biological components of the epigenetic measurement system are determined such that the measured set of epigenetic parameters is identical to the epigenetic parameters of interest as defined in step 1a.

9. A method according to claim 1, wherein the chemical and biological components of the epigenetic measurement system are determined such that the measured set of epigenetic parameters differs from the epigenetic parameters of interest as defined in step 1a differs up to a predefined extent.

10. A method according to claim 9, wherein the difference between the epigenetic parameters of interest and the epigenetic parameters to be measured is estimated.

11. A method according to claim 1, wherein steps a-c are repeated until a predefined data quality is obtained.

12. A method according to claim 1, wherein the selection of epigenetic parameters of interest involves queries in a knowledge representation system that contains known correlations between genetic and/or epigenetic and phenotypic parameters.

13. A method according to any of the claims 1, 3, 8, 9, 10 or 12, wherein the epigenetic parameters of interest are tightened interactively.

14. A method according to any of the claims 1, 3, 8, 9, 10 or 12, wherein the epigenetic parameters of interest are broadened interactively.

15. A method according to claim 12, wherein the epigenetic parameters of interest contain epigenetic parameters with unknown function.

16. A method according to claim 12, wherein the epigenetic parameters of interest contain epigenetic parameters with known function.

17. A computer program product for an epigenetic knowledge generation method, said computer program product comprising the steps of:
a. computer readable program code means for selecting epigenetic parameters of interest;
b. computer readable program code means for designing the chemical and/or biological components of the epigenetic measurement system, wherein the chemical and/or biological components determine the epigenetic parameters to be measured;
c. computer readable program code means for synthesizing the variable chemical and/or biological components;
d. computer readable program code means for measuring the value of the epigenetic parameters using the chemical and/or biological components;
e. computer readable program code means for storing the results obtained by measurement;
f. computer readable program code means for defining a subset of epigenetic parameters of interest based on the measurements;
g. repeating steps a-d.

18. A computer program product for an epigenetic knowledge generation method according to claim 17, where steps a-f are distributed among several locations and wherein the data, the chemical and/or biological components are shipped in a systematic way between the units implementing any of these steps.

19. A computer program product for an epigenetic knowledge generation method according to claim 17, where steps b, c and d are integrated into a single device comprising:
a. the input interface for the design specification;
b. the unit for synthesizing the desired chemical and/or biological components that can be varied in the process and that are determined by the specification of the epigenetic parameters of interest;
d. the unit for measurement;
e. the interface for transmitting the measurement results towards the component that interprets the experimental results.

20. A computer program product for an epigenetic knowledge generation method according to any of the claims 17, 18 or 19, wherein the epigenetic parameters of interest comprise the methylation status of a single or a plurality of CpG dinucleotids in the genome.

21. A computer program product for an epigenetic knowledge generation method according to any of the claims 17, 18 or 19, wherein the epigenetic parameters of interest comprise the methylation status of CpG dinucleotids within selected fragments of selected genes.

22. A computer program product for an epigenetic knowledge generation method according to any of the claims 17, 18 or 19, wherein the epigenetic parameters of interest comprise the methylation status of CpG dinucleotids within promoter regions of selected genes.

23. A computer program product for an epigenetic knowledge generation method according to any of the claims 17, 18 or 19, wherein the epigenetic parameters of interest comprise the methylation status of CpG islands in selected genes.

24. A computer program product for an epigenetic knowledge generation method according to claim 17, wherein the chemical and biological components of the epigenetic measurement system are determined such that the measured set of epigenetic parameters is identical to the epigenetic parameters of interest as defined in step 17a.

25. A computer program product for an epigenetic knowledge generation method according to claim 17, wherein the chemical and biological components of the epigenetic measurement system are determined such that the measured set of epigenetic parameters differs from the epigenetic parameters of interest as defined in step 17a differs up to a predefined extent.

26. A computer program product for an epigenetic knowledge generation method according to claim 25, wherein the difference between the epigenetic parameters of interest and the epigenetic parameters to be measured is estimated.

27. A computer program product for an epigenetic knowledge generation method according to claim 17, wherein steps a-c are repeated until a predefined data quality is obtained.

28. A computer program product for an epigenetic knowledge generation method according to claim 17, wherein the selection of epigenetic parameters of interest involves queries in a knowledge representation system that contains known correlations between genetic and/or epigenetic and phenotypic parameters.

29. A computer program product for an epigenetic knowledge generation method according to any of the claims 17, 19, 24-26 or 28, wherein the epigenetic parameters of interest are tightened interactively.

30. A computer program product for an epigenetic knowledge generation method according to any of the claims 17, 19, 24-26 or 28, wherein the epigenetic parameters of interest are broadened interactively.

31. A computer program product for an epigenetic knowledge generation method according to claim 28, wherein the epigenetic parameters of interest contain epigenetic parameters with unknown function.

32. A computer program product for an epigenetic knowledge generation method according to claim 28, wherein the epigenetic parameters of interest contain epigenetic parameters with known function.

33. A system of epigenetic knowledge generation comprising the steps of:
a. means for selecting epigenetic parameters of interest;
b. means for designing the chemical and/or biological components of the epigenetic measurement system, wherein the chemical and/or biological components determine the epigenetic parameters to be measured;
c. means for synthesizing the variable chemical and/or biological components;
d. means for measuring the value of the epigenetic parameters using the chemical and/or biological components;
e. means for storing the results obtained by measurement;
f. means for defining a subset of epigenetic parameters of interest based on the measurements;
g. repeating steps a-d.

34. The system of epigenetic knowledge generation according to claim 33, where steps a-f are distributed among several locations and wherein the data, the chemical and/or biological components are shipped in a systematic way between the units implementing any of these steps.

35. The system of epigenetic knowledge generation according to claim 33, where steps b, c and d are integrated into a single device comprising:
a. means for the input interface for the design specification;
b. means for the unit for synthesizing the desired chemical and/or biological components that can be varied in the process and that are determined by the specification of the epigenetic parameters of interest;
d. means for the unit for measurement;
e. means for the interface for transmitting the measurement results towards the component that interprets the experimental results.

36. The system of epigenetic knowledge generation according to any of the claims 33, 34 or 35, wherein the epigenetic parameters of interest comprise the methylation status of a single or a plurality of CpG dinucleotids in the genome.

37. The system of epigenetic knowledge generation according to any of the claims 33, 34 or 35, wherein the epigenetic parameters of interest comprise the methylation status of CpG dinucleotids within selected fragments of selected genes.

38. The system of epigenetic knowledge generation according to any of the claims 33, 34 or 35, wherein the epigenetic parameters of interest comprise the methylation status of CpG dinucleotids within promoter regions of selected genes.

39. The system of epigenetic knowledge generation according to any of the claims 33, 34 or 35, wherein the epigenetic parameters of interest comprise the methylation status of CpG islands in selected genes.

40. The system of epigenetic knowledge generation according to claim 33, wherein the chemical and biological components of the epigenetic measurement system are determined such that the measured set of epigenetic parameters is identical to the epigenetic parameters of interest as defined in step 33a.

41. The system of epigenetic knowledge generation according to claim 33, wherein the chemical and biological components of the epigenetic measurement system are determined such that the measured set of epigenetic parameters differs from the epigenetic parameters of interest as defined in step 33a differs up to a predefined extent.

42. The system of epigenetic knowledge generation according to claim 41, wherein the difference between the epigenetic parameters of interest and the epigenetic parameters to be measured is estimated.

43. The system of epigenetic knowledge generation according to claim 33, wherein steps a-c are repeated until a predefined data quality is obtained.

44. The system of epigenetic knowledge generation according to claim 33, wherein the selection of epigenetic parameters of interest involves queries in a knowledge representation system that contains known correlations between genetic and/or epigenetic and phenotypic parameters.

45. The system of epigenetic knowledge generation according to any of the claims 33, 35, 40-42 or 44, wherein the epigenetic parameters of interest are tightened interactively.

46. The system of epigenetic knowledge generation according to any of the claims 33, 35, 40-42 or 44, wherein the epigenetic parameters of interest are broadened interactively.

47. The system of epigenetic knowledge generation according to claim 44, wherein the epigenetic parameters of interest contain epigenetic parameters with unknown function.

48. The system of epigenetic knowledge generation according to claim 44, wherein the epigenetic parameters of interest contain epigenetic parameters with known function.
